(19) United States
(12) Patent Application Publication
(10) Pub. No.: US 2017/0049629 A1
(43) Pub. Date: Feb. 23, 2017

(54) METHOD, KIT, AND TAPE FOR WOUND CARE

(71) Applicants: Spidertech Inc., Toronto (CA); Sunnybrook Research Institute, Toronto (CA)

(72) Inventors: Ray Arbesman, Toronto (CA); Marc Jeschke, Toronto (CA)

(73) Assignees: Spidertech Inc., Toronto (CA); Sunnybrook Research Institue, Toronto (CA)

(21) Appl. No.: 14/831,945

(22) Filed: Aug. 21, 2015

(51) Int. Cl. A61F 13/02; A61F 13/00

(52) U.S. Cl. CPC A61F 13/025; A61F 13/0233; A61F 13/024; A61F 13/0253; A61F 13/00076

(57) ABSTRACT

A wound care tape includes a sterile high-stretch strip of woven material having a first face and an opposed second face. A sterile adhesive is on the first face in a discontinuous pattern, whereby a set of spaced apart adhesive-covered portions of the first face are covered by the adhesive, and a set of spaced apart adhesive-free portions of the first face are free of the adhesive.

### FIELD

The disclosure relates to wound care. More specifically, the disclosure relates to wound care tapes, methods for using wound care tapes, and kits containing wound care tapes.

### BACKGROUND

U.S. Patent Application Publication No. 2013/0334084 (Arbesman) purports to disclose a contusion patch comprising a high stretch therapeutic tape with an adhesive backing. The tape has an anchoring portion that adheres to a contusion and a plurality of fingers that extend from the anchoring portion. The tape is less than about 5 inches in length and about 2 inches in width when unstretched. A frangible release liner covers the adhesive backing of the tape prior to application. The release liner has a greater surface area than the tape, so that an exposed strip of the release liner extends around the tapes perimeter. The release liner is scored at junctions between the anchoring portion and the fingers to allow selective removal during staggered application of the tape.

### SUMMARY

The following summary is intended to introduce the reader to various aspects of the disclosure, but not to define any invention.

According to some aspects, a kit for wound care is disclosed. The kit comprises a package having a sealed and sterile interior volume. A wound care tape is in the interior volume. The wound care tape comprises a high-stretch strip of woven material having a first face and an opposed second face, and an adhesive on the first face in a discontinuous pattern. A set of spaced apart adhesive-covered portions of the first face are covered by the adhesive, and a set of spaced apart adhesive-free portions of the first face are free of the adhesive. The wound care tape does not include an absorbent pad adjacent the first face. A release liner is in the sterile interior volume and adhered to the first face by the adhesive.

The woven material may be woven cotton. The adhesive may be a poly-acrylic adhesive.

At least some of the adhesive-covered portions and some of the adhesive-free portions may be arranged in alternating curved bands. The tape may extend along a longitudinal axis, and at least some of the adhesive-free portions may extend transverse to the longitudinal axis.

The strip of woven material may comprise a pair of adjacent lobes. The tape may be generally lemniscate shaped.

The kit may comprise a plurality of the wound care tapes in the package, and the release liner may be adhered to the plurality of the wound care tapes. The release liner may be perforated along a boundary between each adjacent pair of the wound care tapes. The plurality of the wound care tapes may be arranged in a grid on the release liner.

The strip of woven material may have an unstretched length, and may be elastically stretchable to a stretched length that is at least 1.4 times the unstretched length, or at least 1.5 times the unstretched length.

According to some aspects, a wound care tape is disclosed. The wound care tape comprises a sterile high-stretch strip of woven material having a first face and an opposed second face. A sterile adhesive is on the first face in a discontinuous pattern. A set of spaced apart adhesive-covered portions of the first face are covered by the adhesive, and a set of spaced apart adhesive-free portions of the first face are free of the adhesive. The wound care tape does not include an absorbent pad adjacent the first face.

The woven material may be woven cotton. The adhesive may be a poly-acrylic adhesive.

At least some of the adhesive-covered portions and at least some of the adhesive-free portions may be arranged in alternating curved bands. The tape may extend along a longitudinal axis, and at least some of the adhesive-free portions may extend transverse to the longitudinal axis.

The strip of woven material may comprise a pair of adjacent lobes. The tape may be generally lemniscate shaped.

The strip of woven material may have an unstretched length, and may be elastically stretchable to a stretched length that is at least 1.4 times the unstretched length, or at least 1.5 times the unstretched length.

The tape may be adherable to skin for at least 7 days. The tape may be adherable to skin for at least 7 to 14 days.

Also provided is a use of the tape for treating a wound, and a tape for the use in treating a wound. The tape may be used for treating a wound. The wound may be an acute wound. The wound may be a surgical incision. The tape may be used together with stitches, and/or together with skin glue. Alternatively, the tape may be used alone, without skin glue or stitches.

According to some aspects, a method for treating a wound is disclosed. The method comprises a) adhering a first set of spaced apart adhesive-covered portions of a high-stretch strip of woven material to a patient's skin on a first side of a wound; b) stretching the high-stretch strip of woven material across the wound to a stretched configuration; and c) adhering a second set of spaced apart adhesive-covered portions of the high-stretch strip of woven material to the patient's skin on a second side of the wound while the wound care strip is in the stretched configuration.

During step a), a first set of spaced apart adhesive-free portions of the high stretch strip of woven material may be maintained as not adhered to the patient's skin. During step c), a second set of spaced apart adhesive-free portions of the high stretch strip of woven material may be maintained as not adhered to the patient's skin.

The method may further comprise leaving the strip of woven material adhered to the patient's skin for at least 7 days, for example for 7 to 14 days.

Steps a) to c) may be carried out without applying an absorbent pad between the strip of woven material and the wound.

The wound may be an acute wound. The wound may be a surgical incision.

The method may further comprise applying a second high-stretch strip of woven material to the wound by: a) adhering a first set of spaced apart adhesive-covered portions of the second high-stretch strip of woven material to the patient's skin on the first side of the wound, while maintaining a first set of spaced apart adhesive-free portions of the second high stretch strip of woven material not adhered to the patient's skin; b) stretching the second high-stretch strip of woven material across the wound to a second stretched configuration; and c) adhering a second set of spaced apart adhesive-covered portions of the second high-stretch strip of woven material to the patient's skin on the second side of the wound while the second wound care strip is in the second stretched configuration, while maintaining a second set of spaced apart adhesive-free portions of the second high stretch strip of woven material not adhered to the patient's skin.

The first high-stretch strip of woven material may be applied in a transverse orientation with respect to the second high-stretch strip of woven material. The second high-stretch strip of woven material may be applied to overlap with at least a portion of the first high-stretch strip of woven material.

The method may further comprise, prior to step a), applying stitches to the wound, and/or applying skin glue to the wound. Alternatively, the high stretch strip of woven material may be applied without prior application of any stitches and skin glue.

The high stretch strip of woven material may be sterile. For example, prior to step a), the high stretch strip of woven material may be sterilized.

The high stretch strip of woven material may extend along a longitudinal axis, and when adhered may be stretchable parallel to the longitudinal axis and transverse to the longitudinal axis.

### BRIEF DESCRIPTION OF THE DRAWINGS

The drawings included herewith are for illustrating various examples of articles, methods, and apparatuses of the present disclosure and are not intended to limit the scope of what is taught in any way. In the drawings:

FIG. 1 is a top view of an example wound care tape;

FIG. 2 is a bottom view of the tape of FIG. 1;

FIG. 3 is an enlarged side view of the region shown in box 3 in FIG. 2;

FIG. 4 is a top view of an alternative example wound care tape;

FIG. 5 is a top view of the wound care tape of FIG. 1 on a release liner;

FIG. 6 is a bottom view of the wound care tape and release liner of FIG. 5;

FIG. 7 is a top plan view of a plurality of the wound care tapes of FIG. 1 on a common release liner;

FIG. 8 is a top plan view of the wound care tape and release liner of FIG. 5, inside an example sealed and sterile package;

FIG. 9 is a cross-section taken along line 9-9 in FIG. 8;

FIG. 10 is a schematic top view of a body part with a wound, and with stitches applied to the wound;

FIG. 11 shows the body part, wound, and stitches of FIG. 10, with a first lobe of the wound care tape of FIG. 1 being applied to the skin adjacent the wound;

FIG. 12 shows the body part, wound, stitches, and wound care tape of FIG. 11, with the wound care tape being stretched across the wound;

FIG. 13 shows the body part, wound, stitches, and wound care tape of FIG. 12, with the second lobe of the wound care tape being applied to the skin adjacent the wound;

FIG. 14 shows the body part, wound, stitches, and wound care tape of FIG. 13, with additional wound care tapes applied to the wound.

FIG. 15 shows the body part, wound, and stitches of FIG. 10, with the wound care tape of FIG. 4 applied to the wound; and

FIG. 16 shows the body part, wound, and stitches of FIG. 10, with a plurality of the wound care tapes of FIG. 4 applied to the wound.

### DETAILED DESCRIPTION

Various apparatuses or processes will be described below to provide an example of an embodiment of the claimed subject matter. No embodiment described below limits any claim and any claim may cover processes or apparatuses that differ from those described below. The claims are not limited to apparatuses or processes having all of the features of any one apparatus or process described below or to features common to multiple or all of the apparatuses described below. It is possible that an apparatus or process described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Disclosed herein is a tape for wound care (also referred to herein as a “wound care tape”, or simply as a “tape”), and related kits and methods. The wound care tapes disclosed herein are related to kinesiology tapes. However, it is believed that kinesiology tapes have heretofore not been used directly on broken skin for wound care. That is, kinesiology tapes are traditionally used on intact skin for treating conditions associated with muscles, joints, and/or contusions. Prior art publications have cautioned against using kinesiology tape on broken skin, as it has been believed that applying kinesiology tape to broken skin can cause extensive tissue damage (Stockheimer, 2007). Accordingly, in rare instances where kinesiology tape has been used for wound treatment, care has been taken to apply a pad between the kinesiology tape and the wound (Oka, 2010).

It has presently been determined that kinesiology tapes can be used directly on broken skin for wound care. For example, kinesiology tapes, when cut to a suitable shape and sterilized, can be applied directly to a wound, to hold the wound closed. It has been determined that the use of sterilized kinesiology tapes directly on broken skin for wound care does not necessarily cause tissue damage, such as the extensive tissue damage purported in the prior art. It has further been determined that the use of sterilized kinesiology tapes directly on broken skin for wound care can be beneficial, in that it may be worn for prolonged periods of time, may promote or facilitate healing, may be considered easy to use by physicians, and may be considered to be comfortable by patients.

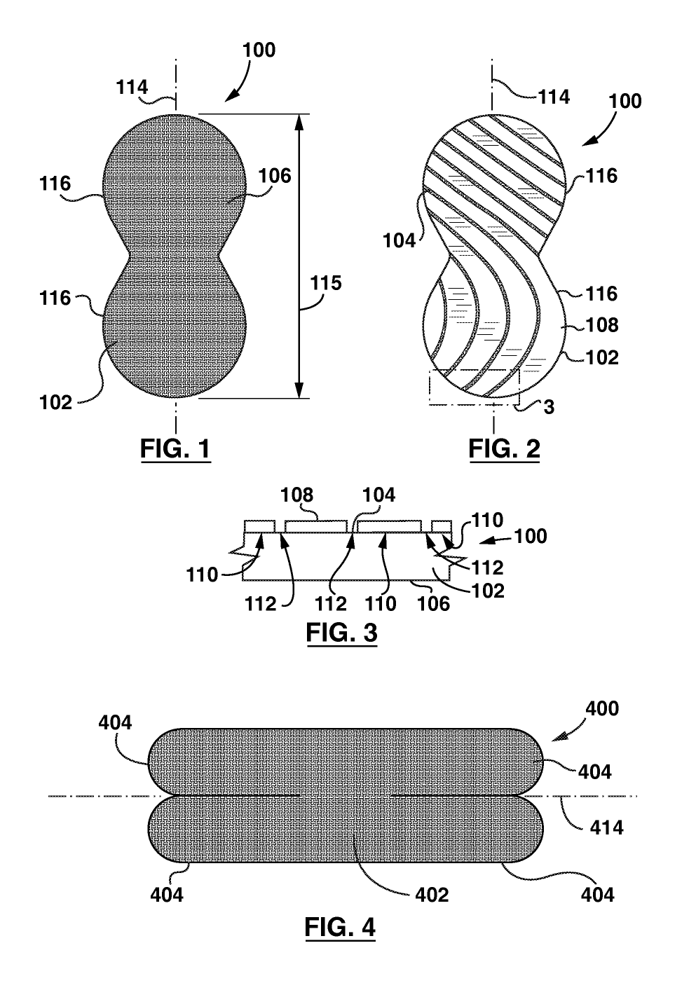

Referring now to FIGS. 1 to 3, an example wound care tape 100 is shown. In the example shown, the wound care tape 100 is made from (i.e. cut from) kinesiology tape, and includes a high stretch strip of woven material (referred to hereinafter as “strip 102”) with an adhesive 108 thereon.

The woven material may in some examples be a woven cotton. The use of a woven material allows for breathability of the tape, which is believed to facilitate and/or promote wound healing, and is also believed to be comfortable for a patient.

As mentioned above, the strip 102 may be high stretch. As used herein, the term ‘high stretch’ indicates that the strip 102 is highly stretchable in at least one direction. For example, the strip may be highly stretchable along its length. For example, the strip 102 may have an unstretched length 115, and may be elastically stretchable to a length that is at least 1.4 times the unstretched length 115, or at least 1.5 times the unstretched length 115. The strip 102 may additionally be highly stretchable along its width, or may be minimally-stretchable or non-stretchable along its width. The term ‘minimally’ stretchable indicates that the strip 102 is stretchable along its width, but less stretchable than it is along its length. The term ‘non-stretchable’ indicates that the strip 102 is not stretchable along its width, or is stretchable to only a negligible extent along its width.

The strip 102 has a first face 104 (shown in FIG. 2) that in use faces the skin of a patient, and an opposed second face 106 (shown in FIG. 1).

Referring to FIG. 2, in the example shown, the adhesive 108 is on the first face 104 in a discontinuous pattern. That is, referring to FIG. 3, the first face 104 includes a set of adhesive-covered portions 110 (i.e. portions that are covered by the adhesive), and a set of adhesive-free portions 112 (i.e. portions that are free of adhesive, so that the first face is exposed). This may allow for the tape 100 to stretch, even when adhered to skin. For example, when the tape 100 is adhered to skin, the adhesive-free portions 112 may readily stretch to accommodate patient movement, while the adhesive-covered portions 110 may remain adhered to the skin and remain generally unstretched, minimally stretched, or less stretched than the adhesive-covered portions. This may allow for the tape to be worn for prolonged periods of time, since the tape may not necessarily peel off the skin when stretched to accommodate patient movement.

Referring still to FIGS. 2 and 3, in the example shown, the adhesive-covered portions 110 and adhesive-free portions 112 are provided in an alternating pattern, so that the adhesive-covered portions 110 are spaced apart from each other, and the adhesive-free portions 112 are spaced apart from each other.

Referring still to FIG. 2, the tape 100 extends along a longitudinal axis 114. At least some of the adhesive-free portions 112 may extend non-parallel to the longitudinal axis 114. Furthermore, at least some of the adhesive-free portions 112 may extend non-perpendicular to the longitudinal axis. For example, the adhesive-free portions may be curved, and may extend generally diagonally with respect to the longitudinal axis 114. This may allow for the tape 100 to stretch and/or accommodate movement in both a lengthwise direction and a widthwise direction (as well as additional directions that have both a lengthwise and widthwise component), even when adhered to skin. In the example shown, the adhesive-covered portions 110 and adhesive-free portions 112 are provided as curved bands that are arranged in an alternating pattern and extend generally diagonally with respect to the longitudinal axis.

The adhesive 108 may be any suitable skin-safe adhesive, such as a polyacrylic adhesive. The adhesive 108 may be long lasting, so that the wound care tape can be worn on skin for prolonged periods of time. For example, the tape may be adherable to skin for at least 7 days. For further example, the tape may be adherable to skin for 7 to 14 days.

Referring still to FIG. 2, in the example shown, the tape 100 does not include an absorbent pad adjacent the first face 104. That is, an absorbent pad is not provided as part of the tape 100. When the tape 100 is applied to a wound, the strip 102 and adhesive 108 may be applied directly to the broken skin and may contact the wound. It has been determined that despite what has been cautioned in the prior art, this does not necessarily cause tissue damage.

Wound care tapes may be provided in a variety of shapes. In the example shown in FIGS. 1 to 3, the strip 102 includes a pair of adjacent lobes 116, so that the tape 100 is generally lemniscate or figure-8 shaped. It has been determined that this shape is particularly useful for wound closure, in that it can be easily and readily applied by physicians, is comfortable for patients, and can remain on a wound for a prolonged period of time (e.g. up to 7 days or longer) and thereby facilitate and/or promote wound healing.

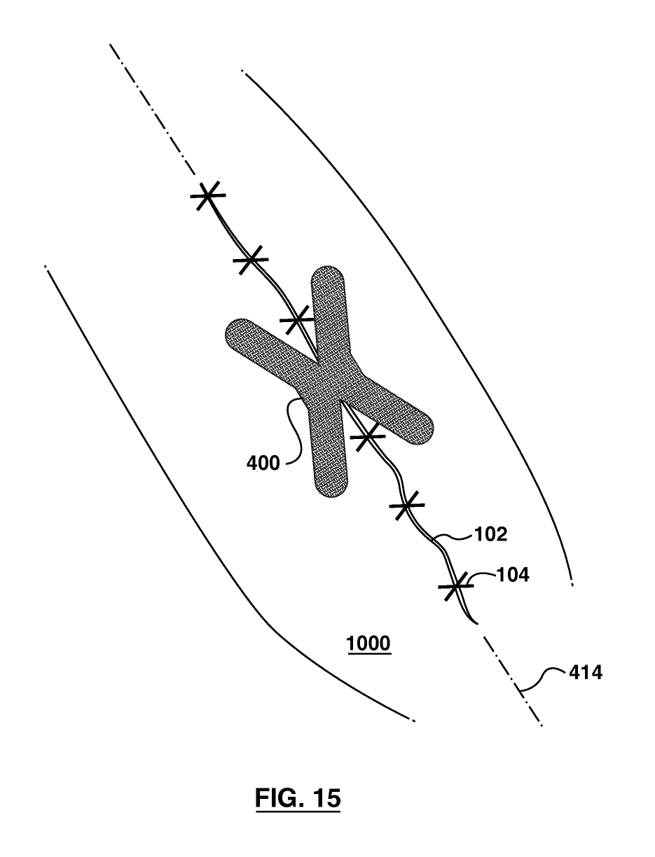

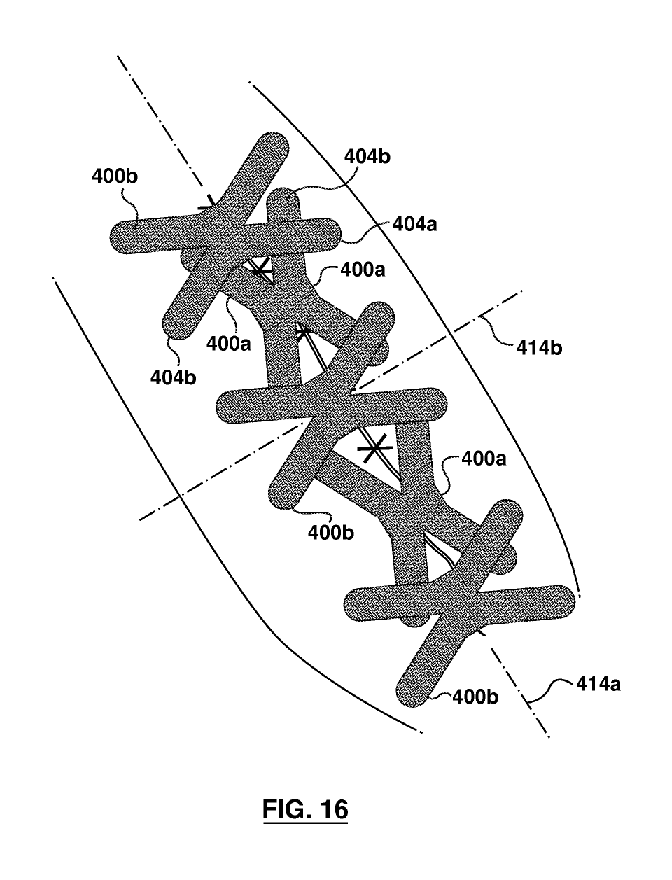

A wound care tape 400 of an alternative shape is shown in FIG. 4. The tape 400 includes a central anchor portion 402, with fingers 404 extending from opposed sides of the anchor portion 402. The fingers 404 are arranged in pairs on opposed sides of the anchor portion. This shape may also be referred to as an X-shape, since when the tape is applied (as described below with respect to FIGS. 15 and 16), adjacent fingers may be separated from each other, so that the tape 400 forms an X-shape.

Wound care tapes may also be provided in a variety of sizes. In some examples, the strip 102 may have an unstretched length 115 of between about 2.5 cm and 7.5 cm, or between about 4 cm and 6 cm, or about 5 cm. In some examples, the strip 402 may have an unstretched length of between about 4.5 cm and 25 cm, or between about 6 cm and 21 cm. In some particular examples, the strip 402 may have a length of about 6.5 cm. In some particular examples, the strip 402 may have a length of about 11 cm. In some particular examples, the strip 402 may have a length of about 20.5 cm.

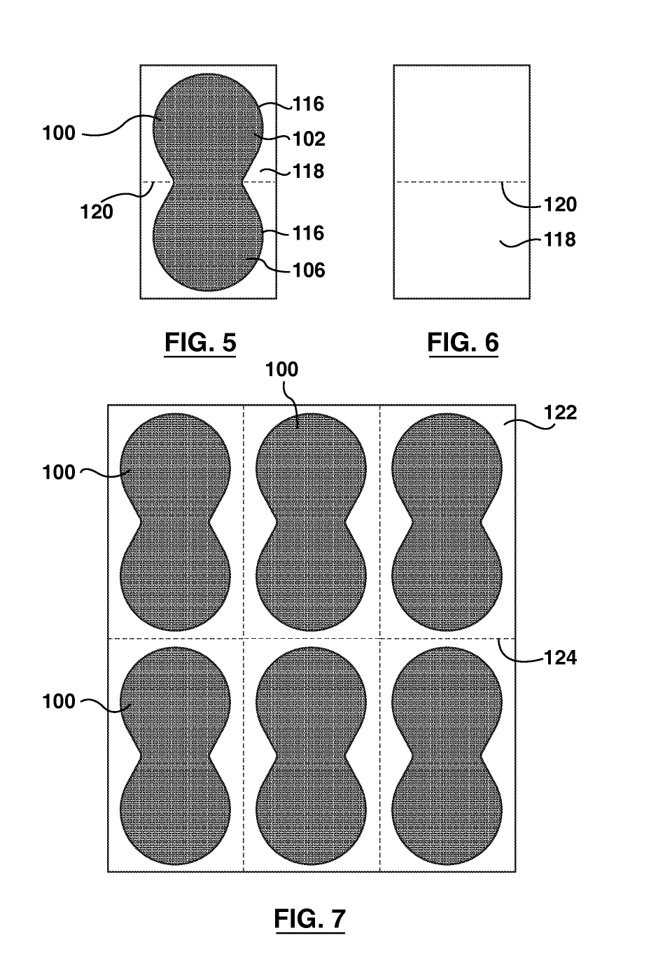

Referring now to FIGS. 5 and 6, the tape 100 may be provided on a release liner 118. The release liner 118 may be adhered to the first face 104 by the adhesive 108. The release liner 118 may be perforated along a boundary 120 between the lobes 116, to facilitate staggered application of the lobes to the skin. For example, the release liner 118 may be severed along the boundary 120, and the resulting release liner portion removed from one lobe 116 of the strip 102. The one lobe 116 may then be applied to the skin adjacent a wound. The remaining release liner portion may then be removed from the other lobe 116, and the other lobe 116 may then be applied to the skin adjacent a wound.

Referring to FIG. 7, in some examples, a plurality of the tapes 100 may be provided, and a common release liner 122 may be adhered to the plurality of tapes 100. The tapes 100 may be arranged in a grid on the release liner 122, and the release liner 122 may be perforated along a boundary 124 between each adjacent pair of the tapes 100.

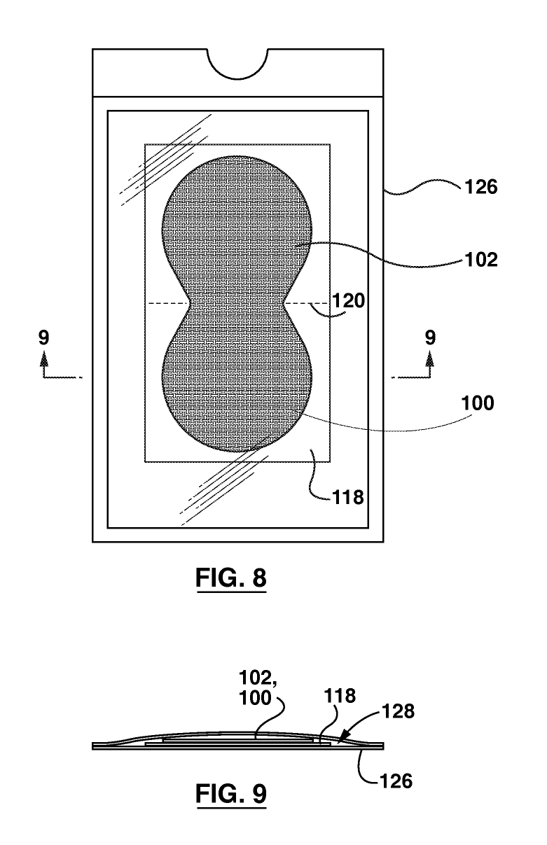

As mentioned above, the tapes disclosed herein (e.g. tapes 100 and/or 400) may be sterile. For example, both the strip 102 and the adhesive 108 may be sterile, in order to be suitable for use on broken skin. Referring now to FIGS. 8 and 9, in some examples, the tape 100 may be provided in a kit that includes a package 126 having a sealed and sterile interior volume 128, with the tape 100 and the release liner 118 in the interior volume 128. The package 126 may be a medical grade peel pouch. The package 126, with the tape 100 and the release liner 118 sealed in the interior volume 128, may in some examples be sterilized by steam sterilization. The package 126 may be opened immediately prior to use of the tape 100.

In alternative examples, a plurality of wound care tapes 100 may be provided in a single package. For example, the release liner 122 and plurality of tapes 100 shown in FIG. 6 may be provided in a sealed and sterile package.

The wound care tapes described above may be used on various types of wounds, including but not limited to acute wounds (e.g. due to accidents), surgical incisions (e.g. for surgical reconstruction of burns or scars), minor wounds (e.g. minor cuts), major wounds, flaps (e.g. free flaps or transpositional flaps), and fresh wounds. The wound care tapes may be used alone, together with stitches, or together with skin glue, depending on the severity of the wound. For example, for some wounds, wound care tapes alone may optionally be used, without skin glue or stitches. For further example, for other wounds, wound care tapes may be used together with stitches and/or skin glue. For example, stitches and/or skin glue may be applied prior to application of the tape.

The wound care tapes may generally be applied in a stretched configuration, so that the elasticity of the tapes aids in holding the wound closed, and supports the skin surrounding the wound.

An example method of using a wound care tape will now be described. For simplicity, the method will be described mainly with respect to wound care tape 100; however, the method may be carried out with other tapes (e.g. tape 400), and the tape 100 may be applied according to other methods.

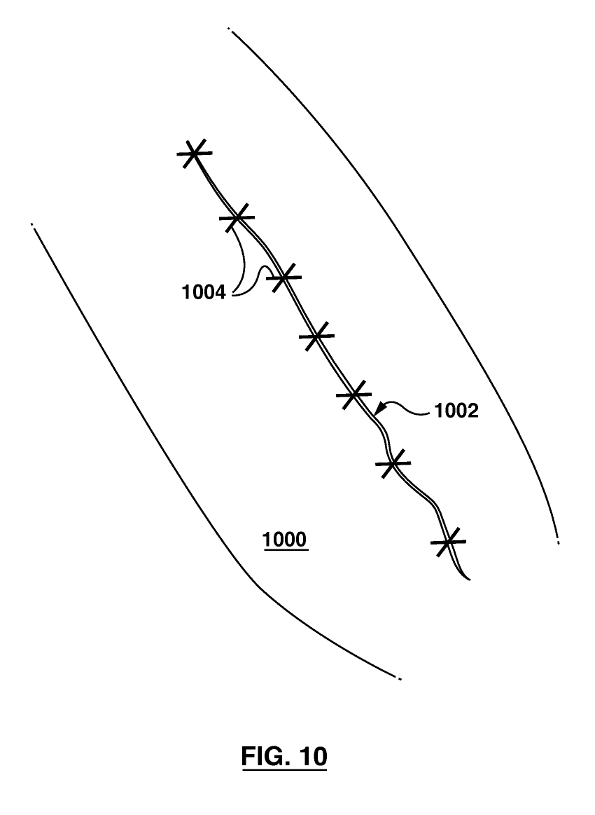

Referring to FIG. 10, a body part 1000 with a wound 1002 is shown. The wound 1002 may be, for example, an acute wound. In the example shown, prior to use of any tapes 100, the wound is closed with stitches 1004. A set of wound care tapes 100 may then be applied to the wound 1002, over the stitches 1004, to assist in holding the wound 1002 closed, and to support the skin surrounding the wound 1002.

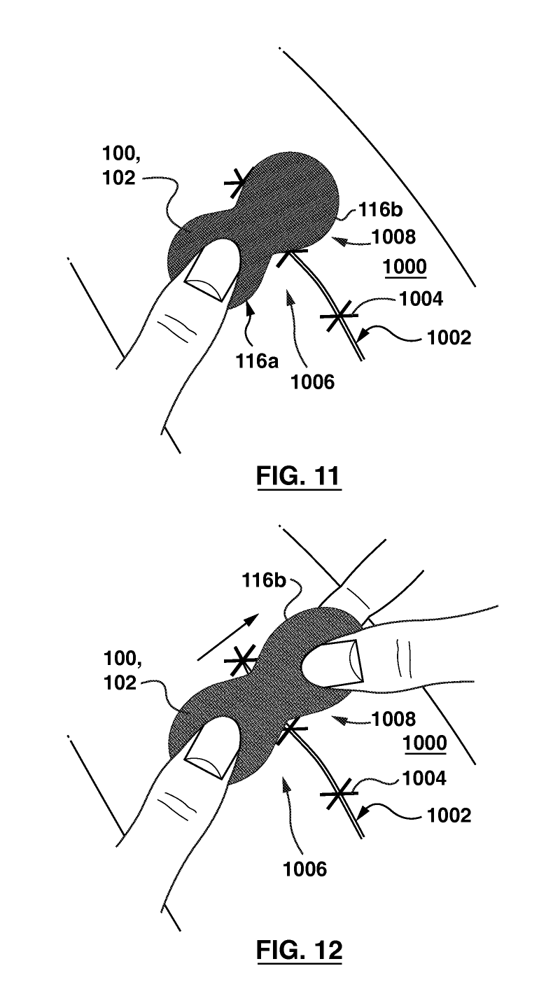

For example, referring to FIG. 11, each wound care tape 100 may be applied by first adhering a first lobe 116a of the strip 102 to the patient's skin on a first side 1006 of the wound 1002. This may be done by pressing the first lobe 116a to the skin on the first side 1006 of the wound 1002, so that the adhesive-covered portions 110 (not shown in FIGS. 11 to 13) on the first lobe 116a (also referred to herein as a “first set” of adhesive covered portions) adhere to the patients skin, while the adhesive-free portions 112 on the first lobe 116a are maintained non-adhered to the patients skin.

Referring to FIG. 12, the strip 102 may then be stretched across the wound to a stretched configuration. For example, the strip 102 may be stretched lengthwise to up to about 1.4 times its unstretched length, or up to about 1.5 times its unstretched length.

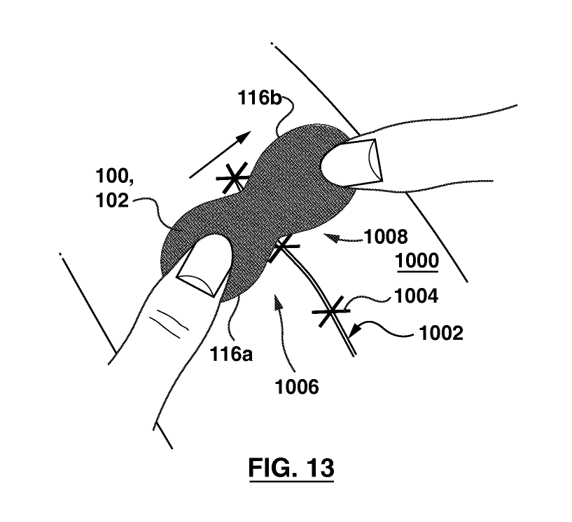

Referring to FIG. 13, the second lobe 116b of the strip 102 may then be adhered to the patient's skin on a second side 1008 of the wound 1002. This may be done by pressing the second lobe 116b to the skin on the second side 1008 of the wound 1002, so that the adhesive-covered portions 110 (not shown in FIGS. 11 to 13) on the second lobe 116b (also referred to herein as a “second set” of adhesive-covered portions) adhere to the patients skin, while the adhesive-free portions 112 (not shown in FIGS. 11 to 13) on the second lobe 116b (also referred to herein as a “second set” of adhesive-free portions) are maintained non-adhered to the patients skin.

The entire strip 102 may then optionally be rubbed against the skin, in order to facilitate adhesion of the entire strip 102 to the skin.

As shown in FIGS. 11 to 13, the tape 100 may be applied without applying an absorbent pad between the strip 102 and the wound 1002. That is, the first lobe 116a is adhered to the skin, the strip 102 is stretched, and the second lobe 116b is adhered to the skin, without applying an absorbent pad between the strip 102 and the wound 1002.

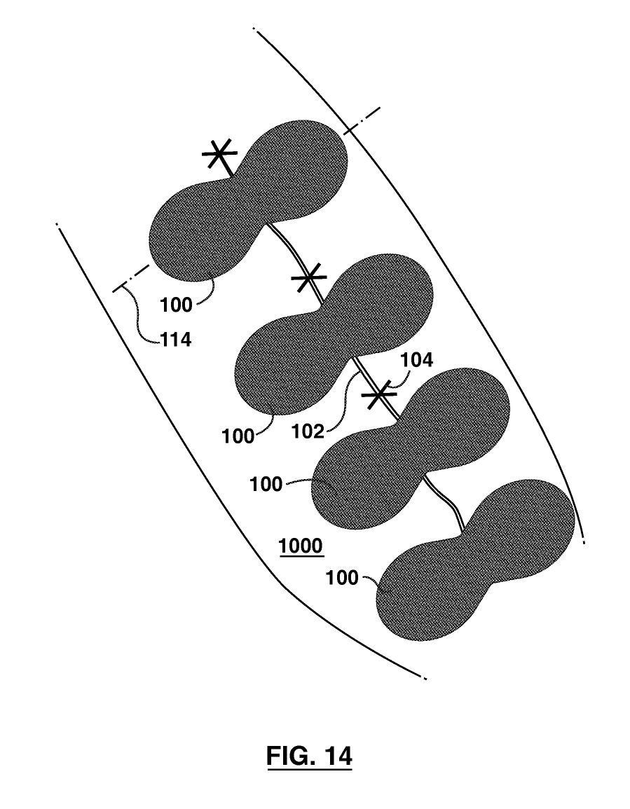

Referring to FIG. 14, the steps shown in FIGS. 11 to 13 may be repeated with additional tapes 100 (e.g. repeated with a second tape through a fourth tape), so that a set of tapes 100 is applied to the wound. For simplicity, the steps described with respect to FIGS. 11 to 13 are not repeated herein for the additional tapes.

The tape 100 may optionally be left adhered to the patient's skin until removal of the stitches 1004. For example, the tapes 100 may be left adhered to the patient's skin for at least 7 days, for example for 7 to 14 days.

In the example described with respect to FIGS. 11 to 14, each tape 100 is applied so that its longitudinal axis 114 (one of which is shown in FIG. 14) extends generally perpendicularly or transverse to the wound 1002. In alternative examples, one or more tapes may be applied so that its longitudinal axis is generally parallel to the wound. For example, referring to FIG. 15, a tape 400 as described above with respect to FIG. 4 has been applied to a wound so that its longitudinal axis 414 is parallel to the wound 1002. For further example, referring to FIG. 16, a set of tapes 400 has been applied to the wound 1002 so that the longitudinal axis 414a of a first set of the tapes 400a is parallel to the wound 1002, and so that the longitudinal axis 414b of a second set of the tapes 400b is perpendicular to the wound 1002. In other words, some of the tapes 400 are applied in a transverse orientation with respect to other of the tapes 400. In this example, the tapes 400 are applied in an overlapping fashion, so that at least a portion of each tape 400 overlaps with at least a portion of another tape 400. More specifically, the fingers 404 of some of the tapes 400a overlap with fingers 404b of other of the tapes (only some of the overlapping fingers 404a, 404b are labelled in FIG. 16).

It is believed that because the adhesive 108 is in a discontinuous pattern on the strip 102, the tape 100 is able to move and stretch as the patient moves, and thereby can remain on the skin for prolonged periods of time. For example, when the tape 100 is adhered and in use, it is stretchable and can accommodate movement parallel to its longitudinal axis 114, and can also accommodate movement transverse to its longitudinal axis 114, without peeling off of the skin. It is further believed that because the strip is made from a woven material, it is breathable and feels generally soft and comfortable to the patient.

In any of the above examples, topical medications may be applied to the skin prior to the applications of the tape. Such topical medications may be used, for example, to protect the skin, enhance adherability of the tapes, and/or provide antiseptic properties. For example, compound tincture of benzoin may be applied to the skin prior to the application of the tapes, in order to protect the skin, enhance adherability of the tapes, and provide antiseptic properties. In other examples, antibiotic creams or ointments may be applied.

While the above description provides examples of one or more processes or apparatuses, it will be appreciated that other processes or apparatuses may be within the scope of the accompanying claims.

### EXAMPLES

Wound care tapes as described above were tested on various wounds, as described in further detail below. The tests were carried out in the Department of Surgery and Plastic Surgery of Sunnybrook Hospital, in Toronto, Ontario, Canada.

Wound care tapes were provided by Spidertech Inc., of Toronto, Ontario, Canada. The wound care tapes included a strip of high stretch woven cotton, with a polyacrylic adhesive on a first face thereof in a discontinuous pattern (as shown in FIG. 2). The tapes did not include an absorbent pad. The tapes were provided in figure-8 shapes (as shown in FIG. 1), as well as X-shapes (as shown in FIG. 4). The tapes were provided on a release liner, in a sealed and sterile package.

### Example 1

The wound care tapes were tested on several patients who had undergone an excision and primary closure of the resulting wound using standard sutures. The wound care tapes were applied across the wound and over the sutures in a stretched configuration, in order to facilitate wound closure. In some cases the wound care tapes were left exposed; in other cases the wound care tapes were covered with gauze. The wound care tapes were left on the wound for 14 days, after which time they were removed in the hospital setting.

It was observed by the attending surgeon that the wound care tapes stayed in place for the duration of the test. It was further observed that the wound care tapes did not cause any maturation, and increased healing.

### Example 2

The wound care tapes were tested on several patients having skin flaps, including transpositional and rotational flaps. The wound care tapes were used in addition to sutures, both to secure the flaps and at the donor site. The wound care tapes were applied across the wound in a stretched configuration, in order to facilitate wound closure. The wound care tapes were left on the wound for 14 days, after which time they were removed in the hospital setting

A strong success rate and excellent outcome were reported by the attending surgeon. No adverse outcomes or complications were reported.

### Example 3

The wound care tapes were tested on patients requiring webspace reconstruction. Webspace was created using advancement and transpositional flaps. The wound care tapes were used in addition to sutures, both to secure the flaps and at the donor site. The wound care tapes were applied to the wound in a stretched configuration, in order to facilitate wound closure. The wound care tapes were left on the wound for 14 days, after which time they were removed in the hospital setting

A strong success rate and excellent outcome were reported by the attending surgeon. No adverse outcomes or complications were reported.

### Example 4

The wound care tapes were tested on small primary cuts (up to 1 cm in length), without any sutures. The wound care tapes were applied across the wound in a stretched configuration, in order to facilitate wound closure.

Excellent cosmetic outcome and excellent healing were reported by the attending surgeon. No adverse outcomes or complications were reported

### REFERENCES

“Management of Scar Tissue”, Kim Rock Stockheimer, University of Wisconsin, LaCrosse Wisconsin, Advance Healing, Summer 2007, page 21
“Kinesio Taping for Skin Wounds”, Kiyotaka Oka, Office Ikuno, September 2010

We claim:

1. A kit for wound care comprising:
a) a package having a sealed and sterile interior volume;
b) a wound care tape in the interior volume, the wound care tape comprising i) a high-stretch strip of woven material having a first face and an opposed second face, and ii) an adhesive on the first face in a discontinuous pattern, whereby a set of spaced apart adhesive-covered portions of the first face are covered by the adhesive, and a set of spaced apart adhesive-free portions of the first face are free of the adhesive, wherein the wound care tape does not include an absorbent pad adjacent the first face; and
c) a release liner in the sterile interior volume and adhered to the first face by the adhesive.

2. The kit of claim 1, wherein the woven material is woven cotton.

3. The kit of claim 1, wherein the adhesive is a poly-acrylic adhesive.

4. The kit of claim 1, wherein at least some of the adhesive-covered portions and some of the adhesive-free portions are arranged in alternating curved bands.

5. The kit of claim 1, wherein the tape extends along a longitudinal axis, and at least some of the adhesive-free portions extend transverse to the longitudinal axis.

6. The kit of claim 1, wherein the strip of woven material comprises a pair of adjacent lobes.

7. The kit of claim 1, wherein the tape is generally lemniscate shaped.

8. The kit of claim 1, wherein the kit comprises a plurality of the wound care tapes in the package, and the release liner is adhered to the plurality of the wound care tapes.

9. The kit of claim 8, wherein the release liner is perforated along a boundary between each adjacent pair of the wound care tapes.

10. The kit of claim 8, wherein the plurality of the wound care tapes are arranged in a grid on the release liner.

11. The kit of claim 1, wherein the strip of woven material has an unstretched length, and is elastically stretchable to a stretched length that is at least 1.4 times the unstretched length.

12. The kit of claim 1, wherein the strip of woven material has an unstretched length, and is elastically stretchable to a stretched length that is at least 1.5 times the unstretched length.

13. A wound care tape comprising:
a) a sterile high-stretch strip of woven material having a first face and an opposed second face; and
b) a sterile adhesive on the first face in a discontinuous pattern, whereby a set of spaced apart adhesive-covered portions of the first face are covered by the adhesive, and a set of spaced apart adhesive-free portions of the first face are free of the adhesive;
wherein the wound care tape does not include an absorbent pad adjacent the first face.

14. The wound care tape of claim 13, wherein the woven material is woven cotton.

15. The wound care tape of claim 13, wherein the adhesive is a poly-acrylic adhesive.

16. The wound care tape of claim 13, wherein at least some of the adhesive-covered portions and at least some of the adhesive-free portions are arranged in alternating curved bands.

17. The wound care tape of claim 13, wherein the tape extends along a longitudinal axis, and at least some of the adhesive-free portions extend transverse to the longitudinal axis.

18. The wound care tape of claim 13, wherein the strip of woven material comprises a pair of adjacent lobes.

19. The wound care tape of claim 18, wherein the tape is generally lemniscate shaped.

20. The wound care tape of claim 13, wherein the strip of woven material has an unstretched length, and is elastically stretchable to a stretched length that is at least 1.4 times the unstretched length.

21. The wound care tape of claim 13, wherein the strip of woven material has an unstretched length, and is elastically stretchable to a stretched length that is at least 1.5 times the unstretched length.

22. The wound care tape of claim 13, wherein the tape is adherable to skin for at least 7 days.

23. The wound care tape of claim 13, wherein the tape is adherable to skin for 7 to 14 days.

24. A method for treating a wound, comprising:
a) adhering a first set of spaced apart adhesive-covered portions of a high-stretch strip of woven material to a patient's skin on a first side of a wound, while maintaining a first set of spaced apart adhesive-free portions of the high stretch strip of woven material non-adhered to the patient's skin;
b) stretching the high-stretch strip of woven material across the wound to a stretched configuration; and
c) adhering a second set of spaced apart adhesive-covered portions of the high-stretch strip of woven material to the patient's skin on a second side of the wound while the wound care strip is in the stretched configuration, while maintaining a second set of spaced apart adhesive-free portions of the high stretch strip of woven material not adhered to the patient's skin.

25. The method of claim 24, further comprising leaving the strip of woven material adhered to the patient's skin for at least 7 days.

26. The method of claim 24, further comprising leaving the strip of woven material adhered to the patient's skin for 7 to 14 days.

27. The method of claim 24, wherein steps a) to c) are carried out without applying an absorbent pad between the strip of woven material and the wound.

28. The method of claim 24, wherein the wound is an acute wound.

29. The method of claim 24, wherein the wound is a surgical incision.

30. The method of claim 24, further comprising applying a second high-stretch strip of woven material to the wound by:
a) adhering a first set of spaced apart adhesive-covered portions of the second high-stretch strip of woven material to the patient's skin on the first side of the wound, while maintaining a first set of spaced apart adhesive-free portions of the second high stretch strip of woven material not adhered to the patient's skin;
b) stretching the second high-stretch strip of woven material across the wound to a second stretched configuration; and
c) adhering a second set of spaced apart adhesive-covered portions of the second high-stretch strip of woven material to the patient's skin on the second side of the wound while the second wound care strip is in the second stretched configuration, while maintaining a second set of spaced apart adhesive-free portions of the second high stretch strip of woven material not adhered to the patient's skin.

31. The method of claim 30, wherein the first high-stretch strip of woven material is applied in a transverse orientation with respect to the second high-stretch strip of woven material.

32. The method of claim 31, wherein the second high-stretch strip of woven material is applied to overlap with at least a portion of the first high-stretch strip of woven material.

33. The method of claim 24, further comprising, prior to step a), applying stitches to the wound.

34. The method of claim 24, further comprising, prior to step a), applying skin glue to the wound.

35. The method of claim 24, wherein prior to step a), the high stretch strip of woven material is sterilized.

36. The method of claim 24, wherein the high stretch strip of woven material is applied without prior application of any stitches and skin glue.

37. The method of claim 24, wherein the high stretch strip of woven material extends along a longitudinal axis, and when adhered is stretchable parallel to the longitudinal axis and transverse to the longitudinal axis.
